(19) United States
(12) Patent Application Publication
(10) Pub. No.: US 2024/0241103 A1
(43) Pub. Date: Jul. 18, 2024

(54) SYSTEM AND METHOD FOR OPTIMIZING STRUCTURAL PROPERTIES OF CONCRETE MIX

(71) Applicant: Joseph Sostaric, Danville, CA (US)

(72) Inventor: Joseph Sostaric, Danville, CA (US)

(21) Appl. No.: 18/415,545

(22) Filed: Jan. 17, 2024

Related U.S. Application Data
(60) Provisional application No. 63/439,516, filed on Jan. 17, 2023.

(51) Int. Cl. G01N 33/38; G06F 16/22

(52) U.S. Cl. CPC G01N 33/383; G06F 16/2282

(57) ABSTRACT

A method is disclosed for mixing and for placing a batch of concrete mix in forms includes assigning a unique serial number to the batch of concrete stored in a database. Admitting measured quantities of concrete ingredients into a mixing vessel, the ingredients including a cement quantity, a water quantity a sand quantity and an aggregate quantity forms the batch. A network collects and stores each of the cement quantity, the water quantity, the sand quantity, and the aggregate quantity in association with the serial number. After curing the batch of concrete mix, the cured batch of concrete mix is tested to derive at least one performance criterion. The performance criterion is stored in the network in association with the serial number. Machine learning is exploited to optimize the ingredients and attributes of the batch based upon the performance criterion.

### FIELD OF THE INVENTION

The present description sets forth a centralized control system for delivering concrete to forms specifically for computer monitoring and electronic sensing of attributes mixed concrete to assure consistent quality.

### RELATED APPLICATIONS

The instant application claims priority to the provisional filing having U.S. Patent Ser. No. 63/439,516 dated 17 Jan. 2023 entitled, “SYSTEM AND METHOD FOR OPTIMIZING STRUCTURAL PROPERTIES OF CONCRETE MIX” which, by this reference is adopted in its entirety as if fully set out herein.

### BACKGROUND OF THE INVENTION

Concrete is the most widely used man-made product on the planet. There are 4 to 5 billion cubic yards (each cubic yard is approximately 2 tons) produced worldwide annually which amounts to more than one ton of concrete for every man, woman, and child on the planet. Although the terms cement and concrete often are used interchangeably, cement is an ingredient of concrete. Concrete is a mixture of aggregates and paste. The aggregates are sand and gravel or crushed stone; the paste is water and Portland cement. Portland cement comprises from 10 to 15 percent of the concrete mix, by volume.

The cement, aggregate, and water are mixed at a batching plant. The pozzolanic chemical reaction is that which causes concrete to get hard. The pozzolanic reaction requires water and is an exothermic reaction giving off heat as the cement in the mix hydrates. The pozzolanic reaction begins to occur when water meets cement and takes place within a defined time frame. The pozzolanic reaction includes a hardening process that continues onward for years even after the concrete “sets” meaning that concrete continues to get stronger even as it gets older. Because of the rapidity of the initial “set,” specifications often call for concrete to be placed within 1-1½ hours from when it is batched. If the manufacturing plant is as little as a half-hour or more from the jobsite, the travel time is subtracted from the window to place the concrete which when so reduced is often short-lived.

There are many environmental or process factors that cause variation in the quality of structures by the pozzolanic reaction, such as the quantity of cementitious material in the concrete mix, the ambient temperature, the temperature of the raw materials, the chemicals in the concrete mix, and the amount of water in the mix, and these factors will determine how and when this reaction takes place. To illustrate this concept, since the process of hydration creates heat, the colder the raw materials and the ambient conditions, the slower the process of hydration occurs and therefore the longer it will take the concrete to set up. How and when concrete sets up (hardens) are of critical importance in optimizing the resulting strength in most construction projects.

Rheology is the branch of physics that studies how materials deform or flow in response to applied forces or stresses. Concrete must be able to properly flow into all corners of the mold or formwork to fill it completely, with or without external consolidation depending on workability class. Catastrophic structural failures may sometimes be traced back to concrete being of unsuitable consistency for the pour resulting in, for example, cold-jointing and honeycombing within the concrete matrix. Therefore, one of the primary criteria for a good concrete structure is that the fresh concrete has satisfactory rheological properties during casting.

There are two types of concrete batch plants. There are ready-mix plants and central-mix plants. Ready-mix plants combine all the materials except for water at the concrete plant. Water is added to a charge of the dry mix and the mixing occurs in the vessel of the ready-mix truck as it rotates on the way to the site. A central-mix plant, on the other hand, combines its ingredients, including water, at a central location and is transported to the construction site as a final ready-made project. Conventionally, in either type of batching plant, there exists a mixer control system (also known as a “batch panel”) which draws on stored information including a recipe number (called mix designs), specified grade of concrete; slump; source and nature of aggregate; and other specifications and according to that information, admits various components into a mixer as ingredients in the intended concrete mix.

The mixing control system effects the measurement of dry ingredients, using weighing hoppers as well as various detection sensors capable of regulating flow of output material. Once requested, the batch panel mixing control system will serve to open and close gates so that precise quantities of each requested ingredient are weighed up properly and discharged in the proper sequence into a plant mixer (known as a pre-mixer) or directly into a ready-mix truck.

Variability in water in the mix, however, can defeat the control the mixing control system imparts. Unmetered water sneaks in with the remaining ingredients. All sources of water, whether metered or not, contribute to hydration in the resulting mix. Water can be added to the mix from multiple sources including:
1. Water that is either weighed or metered into a load;
2. Moisture that is carried in the sand (this can be up to half of the total water required in a given mix design); and
3. Moisture residing in the coarse aggregate.

A mixing controller system actuates devices to meter ingredients, actively measuring the ingredients as admitted into the mixer. The person controlling the mix at what is known as a batch panel is commonly known as the batchman or plant operator. The batchman is initiates and oversees the mixing controller system as it weighs up materials and, in turn, loads trucks at a concrete batch plant. Much as in baking, the resulting mix will vary in accord with the skills of the batchman, the ambient temperature, and the ambient humidity. An important part of the batchman's job is to “fine tune” or adjust the loads prior to discharging the loads into the truck. The batchman will pull or add water based on his or her knowledge of:
Previous mix or batch performance;
Weather conditions, humidity and temperature;
Travel time to the jobsite;
Plant feedback;
Driver feedback;
Mix application;
Customer request;
Residual water remaining in the drum of the truck; and
Changes in the moisture content of the sand or coarse aggregate.

By conventional means, the batchman relies as much upon art as upon science. A batchman may also be aware of a particular cement brand being used and the attributes of a resulting concrete mix based upon such a brand. One such attribute of a particular cement, is called an “optimum sulfate content,” or “optimum gypsum content.” Sulfate in cement, both the clinker sulfate and added gypsum, retards the hydration of the aluminate phase. If there is insufficient sulfate, a flash set may occur; conversely, too much sulfate can cause false setting. The solubility of the sulfate, and therefore its availability to produce retardation or to cause a false set (also known as “plaster set”) depends on the starting materials used and the temperature in the cement mill. Much of the clinker sulfate is present as alkali sulfate, usually in the form of aphthitalite (Na₂SO₄), arcanite (K₂SO₄) or calcium langbeinite (K₂Ca₂(SO₄)₃), or a mixture of some or all of these. Sodium and potassium sulfates can affect strength development by increasing the alkalinity of the pore fluid; when a sulfate ion reacts with aluminate phase to make ettringite and is removed from solution, the loss of the sulfate ion is balanced by the creation of two hydroxyl ions.

Variability in performance of concrete mixtures is not conventionally well-controlled relying as it does on the skill of the batchman. The system, itself, has no memory outside of that of the batchman. What is needed in the art is a more regimented system responsive to environmental conditions, nature of the cement mix and water available to the mix.

### SUMMARY OF THE INVENTION

A method is disclosed for mixing and for placing a batch of concrete mix in forms includes assigning a unique serial number to the batch of concrete stored in a database. Admitting measured quantities of concrete ingredients into a mixing vessel, the ingredients including a cement quantity, a water quantity a sand quantity and an aggregate quantity forms the batch. A network collects and stores each of the cement quantity, the water quantity, the sand quantity, and the aggregate quantity in association with the serial number. After curing the batch of concrete mix, the cured batch of concrete mix is tested to derive at least one performance criterion. The performance criterion is stored in the network in association with the serial number. Machine learning is exploited to optimize the ingredients and attributes of the batch based upon the performance criterion.

There is significant variability in the concrete production and delivery processes caused by the failure to monitor the concrete throughout its plastic state (before it hardens) and manual adjustments made by multiple individuals throughout the production, delivery, and placement of the concrete. By gathering data in real-time and by maintaining a database of historical performance of concrete, a network can be built that takes all these data sources and develops algorithms and artificial intelligence to make quicker, more accurate adjustments to concrete leading to reduced standard deviation of produced product, reduction in costs to produce, and a reduction in the raw materials needed thus the carbon footprint generated from producing ready-mix concrete.

The concrete industry is underserved with the current batch panels on the market, in that quality of the resulting concrete mix is heavily dependent on the judgement an operator uses to formulate the batch. Generally, the data provided to the operator is insufficient to make appropriate adjustment to the formulation. The inventive system, by monitoring and collecting more germane data throughout the formulation process, allows an automated batch panel to make accurate batch adjustments to optimize qualities of the resulting mix. This would be of significant benefit to the concrete producer and would allow substantial savings and benefits in the following areas:
Reduced raw material costs. Variability in mix performance could be reduced allowing a reduction in overdesign necessary to accommodate for the variability in the batching process.
Reduced carbon footprint. By reducing performance variability, over-design can be reduced allowing for the reduction of cement in the concrete mixture.
Better documentation. The current process used by most ready-mix producers is to make final adjustments to the mix outside of the batching process. Cement, aggregate, or water is added at the jobsite and the plant and is often not recorded properly in a manner to give information to the batchman.
Reduced human errors in batching process. Batchmen can make incorrect adjustments based on lack of knowledge or limited/incorrect information supplied to them.
Reduction in delivery time savings. Adjusting a mix takes time. This additional time adds to the delivery costs.
Fewer concrete problems associated with variable concrete performance. Excessive water in a mix causes problems like increased permeability, excessive cracking, and reduced strength. Too little water or admixture can cause consolidation issues such as voids in the finished product. Informed by internal calculations, the inventive control board would generate a batch slump target and make the adjustments to the mix design (within prescribed parameters) to batch the concrete to that prescribed level of consistency.

Some data that would be collected with each concrete batch include the following data:
Ambient temperature.
Ambient humidity.
Wind speed (cooling forms by convection).
Raw material temperatures (temperatures of sand, aggregate, cement and water are recorded and in some embodiments, an additional temperature sensor in the fill pipe to record cement temperature at the time it is blown off into the silo).
Rainfall (important with plants that have open top aggregate bins) recorded either at the plant or by the National Weather Service.
Aggregate bin level sensors to assure correspondence between recipe aggregate volumes and those incorporated in the mix.
Hydraulic pressure or amp/watt meter readings (either value could be used in an alternate means to express concrete slump or spread before the concrete mixture is discharged into the ready-mix truck, in the truck, or in the concrete pumper or all of them).
Concrete temperature throughout the process (pozzolanic reaction is exothermic so knowing the temperature of the mix relative to the ambient temperature gives insight into the resulting concrete structure).
Travel time to jobsite is reflective of a state of “doneness” relative to the batch of mix.
Projected discharge time is also reflective of a state of “doneness” relative to the batch of mix.

In addition, in some embodiments, data would be collected throughout the delivery process (from those trucks equipped to collect additional data) including:
Slump/spread measurements at different time variables;
Ambient temperature throughout the transit trip;
Concrete temperature throughout the transit trip;
Air content measurements:
Drum revolutions; and
Drum speeds.

Such data as collected is recorded and stored in association with the relevant and serialized concrete mix batch. The data would be used to formulate and, ultimately, to exploit in algorithms designed to optimize the exact mix quantities to be batched so that the concrete would arrive at the prescribed slump or consistency and would provide the performance the application requires. Adjustments would be made automatically and instantaneously for variables such as travel time, concrete temperature and ambient temperature as these data are collected. Unlike current means and methods, no manual adjustments would be required after the machine learning process converges upon a solution for a set of the measured variables.

### BRIEF DESCRIPTION OF THE DRAWINGS

Preferred and alternative examples of the present invention are described in detail below with reference to the following drawings:

FIG. 1 depicts deployment of sensors along nonlimiting path of a batch of concrete mix from the batching plant to final pours of that concrete mix;

FIG. 2A depicts a first embodiment of a data network configured to collect data as to a particular batch of concrete mix as it passes from the batching plant to final pours of that concrete mix;

FIG. 2B depicts a second embodiment of a data network configured to collect data as to a particular batch of concrete mix as it passes from the batching plant to final pours of that concrete mix;

FIG. 3 sets out the standardized prior art configuration of an Electronics Product Code (EPC) as exists on RFID cards;

FIG. 4A depicts a nonlimiting example of an embodiment of a data exchange device used to allow interaction between two stations as depicted on FIGS. 2A and 2B after authentication using RFID tags as security passes;

FIG. 4B depicts a state machine diagram showing the authentication using RFID tags as security passes;

FIG. 5 depicts a nonlimiting example of a master server upon which machine learning algorithms operate upon the actions exerted upon a batch of concrete mix to optimize each to produce the highest quality concrete pour.

### DETAILED DESCRIPTION OF THE PREFERRED EMBODIMENT

An observation that underlies the whole of this invention is that though concrete has been a building material for over two thousand years; the pozzolanic reaction is still only vaguely understood because comprehensive data has not been collected. At each stage, from classifying aggregate, mixing the paste, and, then, to actual pouring and hardening, information about the progress and makeup of the materials undergoing the pozzolanic reaction is sensed, time-stamped, batch-stamped, and stored for correlation, review, and improvement by machine learning.

Advantageously, the presently preferred embodiment relies upon many sensors that are commonly available as configured for various industrial processes such as those for temperature, humidity, wind speed, volume, weight, amperage, and pressure. Even where not sensed, reliable data, however, such as localized rainfall and other weather conditions can be downloaded from such as the National Weather Service, a division of the National Oceanic and Atmospheric Administration at, for example: http:/weather.gov/gis/ and related pages. As an alternative embodiment, the system might rely upon meteorological observations taken at each of the batch plant and, optionally, at the construction site. As neither the method for observing weather nor those for collecting the industrial process metrics mentioned above are novel, they are not, here, explained in depth as the same are well-known in the art.

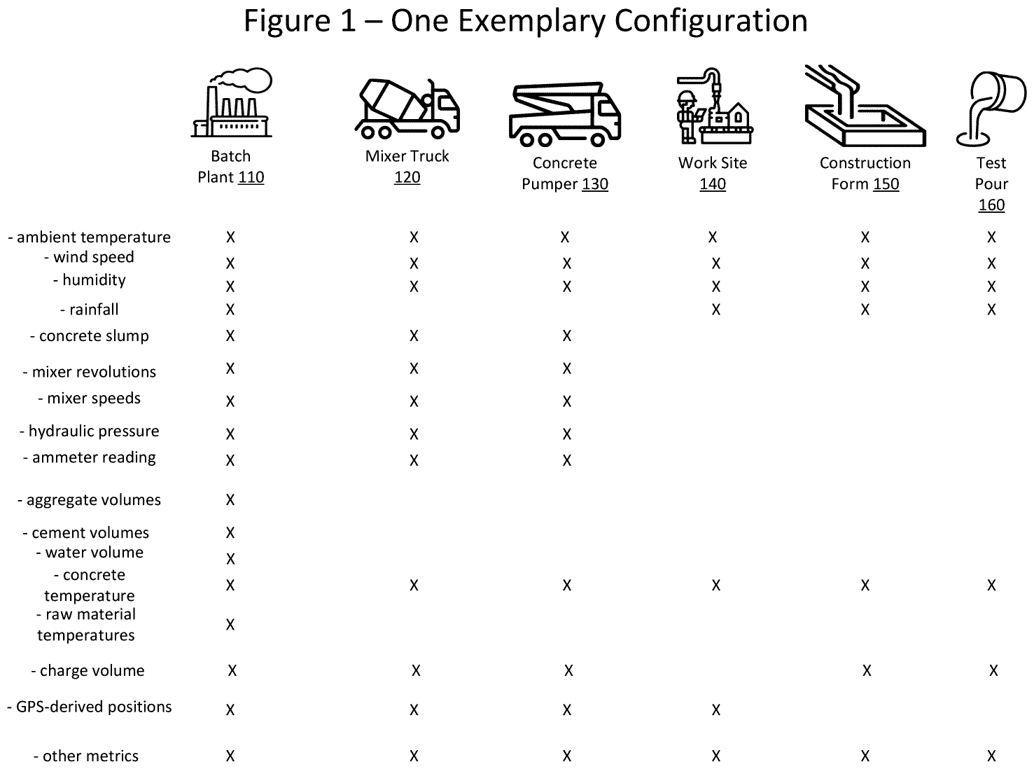

Referring now to FIG. 1, one non-limiting example of monitoring according to the invention is set out. In this nonlimiting embodiment, one of the first measurements made at each site, the batch plant 110; the mixer truck 120; the concrete pumper 130; the work site 140; and each of the construction form 150 and the test pour 160, the ambient temperature is periodically recorded on designated intervals.

The American Concrete Institute's (ACI) technical publications are an excellent resource that provide insights regarding the plethora of effects of environmental factors on concrete. The ACI covers a variety of subtopics, ranging from extreme temperatures, humidity levels, wind velocity, natural disasters, saltwater, and freshwater's effects on concrete to give contractors and concrete industry professionals accurate and up-to-date information. Concrete is a delicate material. It is not only affected by extreme temperatures, but also by humidity levels, as well as the velocity and intensity of the wind. High wind causes the surface of the concrete to dry at a quicker rate than the rest of the concrete. This differential set of the concrete can lead to concrete surface cracks know in the industry as plastic shrinkage cracking. Working with concrete demands skill and patience. It requires both the expertise of those working with concrete, as well as the cooperation of environmental factors to produce a smooth, strong, properly cured structure. For this reason, each of these three meteorological factors are measured in the preferred embodiment of the invention at each station: the batch plant 110; the mixer truck 120; the concrete pumper 130; the work site 140; and each of the construction form 150 and the test pour 160.

The ‘slump’ of concrete refers to the consistency of fresh concrete before it sets—the higher the slump rating, the more fluid the concrete is. Slump is a measurement of the workability or consistency of concrete. In other words, it measures how easy the concrete is to push, mold and smooth out. Accordingly, its slump rating indicates what construction application the concrete is good for. The higher the slump, the more workable the concrete. If the slump of concrete is too low, it won't shape very easily. If it is too high, one runs the risk of having the gravel, sand and cement settle out of the mixture, making it unusable. It is known that the addition of too much water to the concrete mix will give the mix an excessive slump which, in the resulting cured concrete will cause either or both of excessive cracking or reduction in compressive strength. Slump has proven to be one of the best predictors of performance of a concrete mix as it hardens. As such, even an embodiment of the inventive system that measured and recorded only slump of a concrete mix at the batch plant 110; the mixer truck 120; and the concrete pumper 130 would yield more information and, thus, more consistency among batches of concrete than the conventional system.

In each of the batch plant 110; the mixer truck 120; and the concrete pumper 130, there exist, in the conventional configuration of each, augers, mixing paddles, rotating helical fins in a mixing drum, to work the concrete mix. In various embodiments, one sensor the invention comprises additionally measures other of the variables that determine the interval a concrete mix will remain suitably plastic for pumping. The effort exerted to work that concrete mix is measurable. In such embodiments, not only can the invention judge the instantaneous workability of the concrete mix but can additionally predict the expected workability of the mix during the process of pumping concrete through a hydraulic pump.

Based upon the measured effort (in the case of electrical energy expended the measurement of watts or in the case of hydraulic motors, hydraulic pressure and flow) exerted on the concrete mix. Using an embedded micro-controller 411, embodiments of the invention calculate and record one of these metrics as a measurement for the workability or “slump” of the concrete. Various embodiments of the invention also associate other measured information about the mix along with other stored data points including temperature. These are communicated to a master server 500 which can use algorithms working on the sent information to determine if the concrete is beginning to “set up” (a condition caused when the cement in the concrete begins to hydrate). As an added benefit, monitoring the process closely allows the master sever 500 to develop a “map” indicating states of hydration while pumping in order to optimize the state of concrete as it is pumped or, also, optionally, to alert an operator when a quantity of cement mix falls outside of a recommended range of measured slump so the hopper and pipes can be suitably emptied before the concrete can permanently ruin component parts of the pumping assembly.

In each instance wherein working of the concrete occurs, whether within the batch plant 110; the mixer truck 120; and the concrete pumper 130, effort expended in the turning of vanes, paddles, or augers whether by either hydraulic or electrical motivation, sensors measure effort and from effort slump is calculated. Thus, slump might be measured by this form of logical induction rather than by direct observation of slump.

In electrical systems, where measured drawn watts are used to measure the effort expended to mix the concrete mix as a surrogate for directly measuring slump, there are two relevant metrics, i.e. voltage, the force required to move electrons and amperage or current. Current is the rate of the flow of charge per second or electrical current through a material to which a specific voltage is applied. By taking the voltage and multiplying it by the associated current, the power, measured in watts, can be determined. P=V*I where power (P) is in watts, voltage (V) is in volts and current (I) is in amperes. A watt (W) is a unit of power defined as one Joule per second.

The current through the motor is proportional to the torque produced by the motor. Normally the back electromotive force created by the speed of the motor opposes the voltage being applied and limits the amount of current the motor draws and thus its torque. Thus, by measuring the current draw of an electric motor under otherwise known conditions, the operator can relate the current draw at the electric motor driving the pump, one might measure the workability or slump of concrete being agitated. Therefore, where electricity is the motivating force, slump will be in proportion to the work performed by moving augers, vanes, or paddles. Both that and the resulting rotational speed of those moving augers, vanes, or paddles.

In another embodiment, a pressure sensor in the “high” side of the hydraulic line pair can similarly measure workability of the concrete mix in the hopper. Here, the analogy to the electrical system is evident. Sensing pressure and rotational speed will result in a measure of the slump of concrete being worked by the subject vanes, paddles, and augers. The present invention monitors current draw at a known rotational speed to determine the slump of concrete within the hopper. In another embodiment, a pressure sensor in the “high” side of the hydraulic line pair can similarly measure workability of the concrete mix in the hopper.

Deviations in slump as might be observed in values predicted based upon ingredients signal the computer the batchman uses to mix the concrete to indicate water in the mix that was not measured at the hoppers. Measuring slump at the mixing plant yields a warning that unmeasured ingredients are incorporated into the concrete mix. The presence of unmeasured water must be reckoned and compensated for in the optimal mixing of the concrete. For example, moisture in sand changes the amount of water in the mix beyond that metered into the mix. Similarly, more sand can be incorporated as included dust when considering the sand included in the aggregate bin. In controlled circumstances metered amounts of materials should produce a mix with a predicted slump. Where the measured slump deviates from the predicted slump, the magnitude of the deviation can, in most instances, be repaired at the mixing computer at the batch plant 110. For example, where more moisture is introduced with the metered sand, the slump is going to be more than the predicted value. In response, more of the remaining components, sand and cement can be added in a quantity determined by the measure of increased slump. By measuring how much the slump is increased, the amount of additional water can be derived by a known algorithm. This batch is then serialized and the specific “tweaks” to the mix are stored in association with the batch serial number.

There are other instances where the measured quantities in the mix can cause deviation from the anticipated slump value. Added sand residing in a rock bin would cause the resulting mix computer weighs up to include more sand and less rock than the mix design requires. This, too, is detectable. In embodiments of the invention, the computer might be programmed to either make an automatic adjustment (as might be the case with moisture adjustments) or alert the operator of the unexplained variance (as would be the case with weighing up the wrong material). Here too, the quantities of components in the mix, each batch identified by serial number, are stored on the server memorializing the measured mix in terms of deviations from the ideal mix.

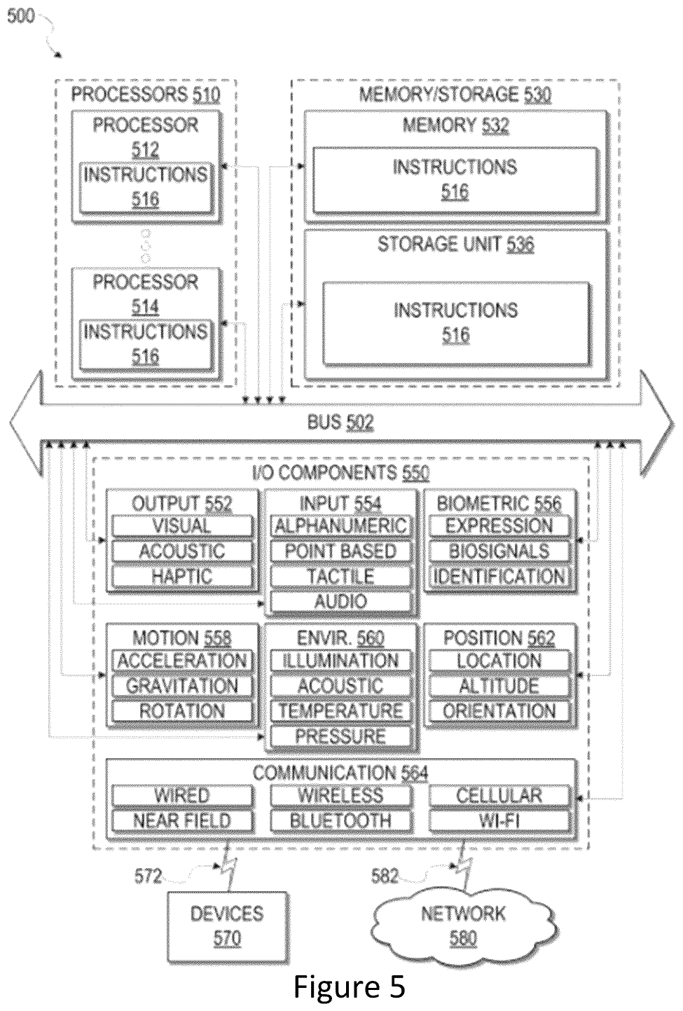

In either embodiment, close monitoring the of the pozzolanic process allows enables a controller 500 (FIG. 5) to process monitored data the system collects and with that data to develop an algorithmic “map”. Theses algorithms predict states of the concrete mix in response to the measured environmental conditions in which the concrete mix matures. Embodiments employing machine learning optimize this mapping of data to characterize each serialized batch of concrete. With this map and the processor 500 (FIG. 5) allows the invention to optimize the state of each batch concrete as it is pumped into receiving trucks, or, also, to alert an operator when a quantity of concrete mix falls outside of a recommended range. In this same manner, an operator of embodiments of the invention can know how nearly any batch in any of a drum, hopper or pipes falls within tolerances. Even where a batch of mix falls outside of tolerance, the truck carrying that batch can be suitably emptied of setting concrete before the concrete can permanently ruin any component parts of the relevant assembly.

Referring to FIG. 1, in the presently preferred embodiment, the temperature of the concrete mix is taken with periodic regularity at each locus in the process, i.e. at the batch plant 110; the mixer truck 120; the concrete pumper 130; the work site 140; and each of the construction form 150 and the test pour 160. As stated above, measuring the temperature of the concrete mix is relatively rudimentary in each of the stations except at the construction form 150 and the test pour 160, as discussed immediately below. On the other hand, in every vessel such as the mixing vessel at the batch plant 110 or within the mixing drum of the mixer truck 120, process temperature technology is mature and may be readily taken, time-stamped and recorded.

Concrete must remain relatively warm for it to set and cure properly. It needs to be at least 40 degrees Fahrenheit while poured and at least 50 degrees when curing. The demands for a supportive environment are so strict that sometimes an outside heating source is necessary to project warm air near the concrete during cold weather conditions. Recording temperature of the poured concrete can assure that the curing occurs under proper conditions or, where deviation is detected, corrective measures may be taken.

In the presently preferred embodiment, a fully embedded wireless device that can be used to check temperature from fresh mixed to the cure-hardened stages at either of the construction form 150 or the test pour 160. In the presently preferred embodiment, wireless sensors are installed within the concrete formwork, typically on the rebar before concrete is placed. The sensor is then connected to a mobile transponder which records instantaneous temperatures in real-time with time and, again in the presently preferred embodiment, with data collected at a global positioning system (“GPS”) location receiver or, where used on a LoRaWAN, the geographic location of the central gateway nearest the transducer. The temperature sensors are configured to work in many extreme conditions and can easily handle temperature values of up to 170 degrees Fahrenheit (as is the case for mass concrete foundations, retaining walls, or dams). In one preferred embodiment, the sensor collects temperature sensor data at designated discrete time intervals (normally 30 minutes or less). While wired connections will work, wireless have proven to be more accurate and when encased in the hardened concrete can continue to function for as long as ten years based upon conventional devices.

While a presently preferred embodiment is set out here, still other metrics might also be derived from other sensors not mentioned here. This invention is not limited to the sensors set out herein, but as reflected in FIG. 1, other further sensors might be advantageously employed.

Once the various data are collected in a time-stamped format and associated with the relevant serialized concrete batch, it is necessary to convey that data to a single location to analyze that batch of concrete and from that analysis, take such measures to characterize the batch. Machine learning is not possible without collecting and grouping such data. Therefor some network is necessary. That the mixer truck 120, the concrete pumper 130, and, to a limited extent, the work site 140, are dynamically moving, thus, this fragmented network presents a challenge of greater depth than might be present in an entirely static network. Much of the inventive structure is mobile such as the concrete mixer truck 120 and the concrete pumper 130. Also, work sites 140 change with each contract and forms for each of the construction form 150 and the test pour 160 vary from phase to phase of the construction. For this reason, the topography of the network must meet rigorous and distinct standards. For example, where a batch plant 110 serves a variety of contractors with several mixer trucks 120, the identity of the contractor, the mixer truck, the batch and the work site 140 will each need to be tracked. For that reason as well, aspects of the inventive network are novel. Two embodiments are set out here, one by which data are collected from the various stations and returned in bucket brigade fashion to the batching plant 110 and one in which stations communicate with a data center 270, some by mobile means such as, by way of nonlimiting example, either by LoRaWAN or cellular networks.

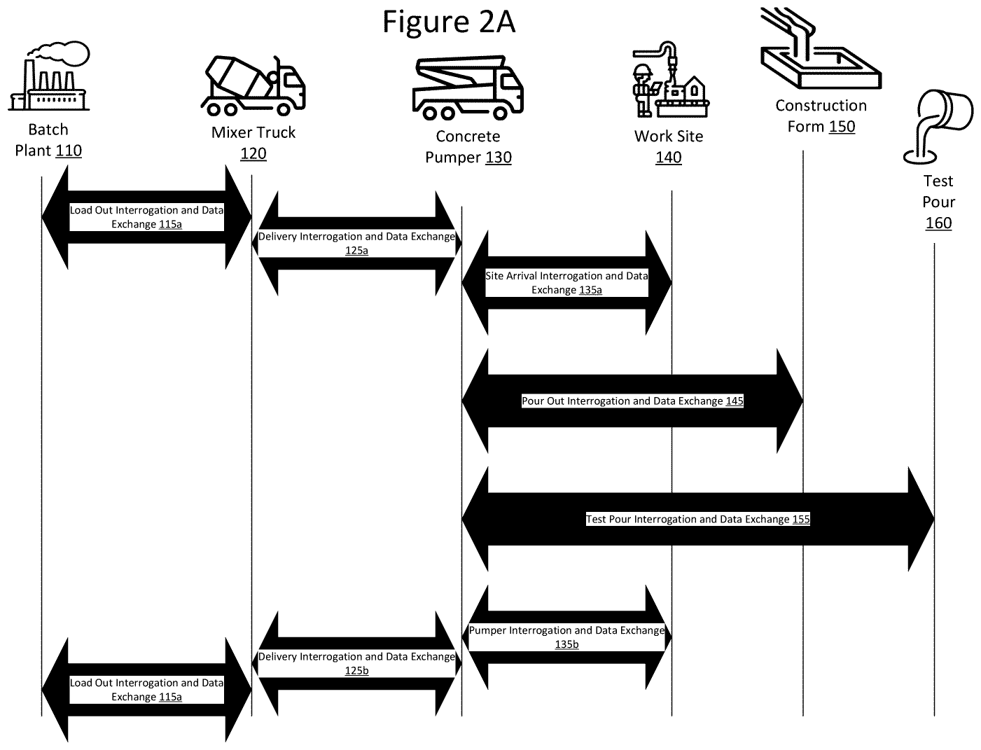

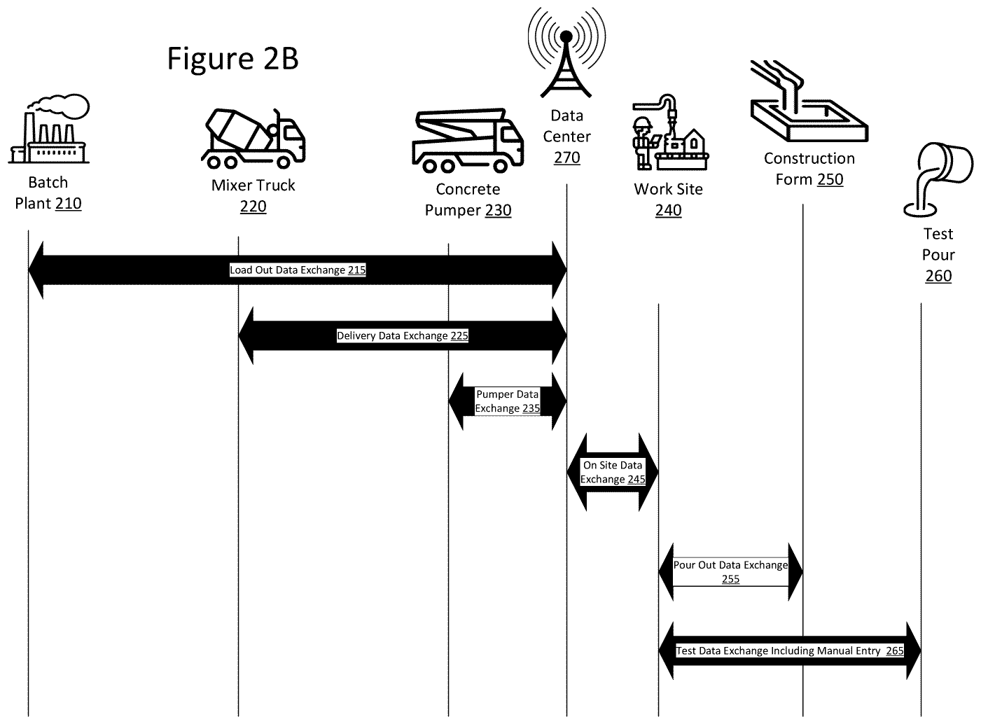

As stated, there are two preferred embodiments of the invention wherein the collected data are retained for processing. In the first of these two embodiments, that depicted in FIG. 2A, data are ultimately retained on a server at the batch plant 110 as each element passes the data back to the batch plant 110. In the second of the preferred embodiments, that depicted in FIG. 2B, all data is retained at “cloud-based” (meaning at a server farm accessible through the Internet) exploiting “IoT” or Internet of things technology to retain the data at a centralized location, in this case represented as a data center 270. Transmission of data will be explored in the succeeding descriptions of data movement based upon which of the two presently preferred embodiments are exploited to effect the goals of the invention.

The discussion of the two presently preferred embodiments that ensues are based upon certain presumed parameters. These presumed parameters are presented for illustrative purposes and the actual embodiment of the invention might include slight variations without departing from the spirit or intent of the invention. The supply chain set out in each of the discussions of the two embodiments depicted, respectively, in FIGS. 2A and 2B, may be divided, roughly, into three phases: off-site (production) status referring to the production of concrete at the batch plant; delivery (shipping) phase, in this discussion depicted as each of the mixer truck 120 and the concrete pumper 130 (though in some envisioned embodiments, the concrete pumper 130 might only be implicated in the supply chain when the concrete pumper 130 is active at the construction work site 140, however, in the instant discussion, the concrete pumper 130 is grouped in the transportation phase), and on-site (work site 140, construction form 150 and the test pour 160) phase. The final phase, the on-site phase of the material logistics starts by receiving the materials on the construction job site and the concrete pumper 130 is discussed herein as being encompassed in the transportation phase rather than the on-site phase.

To allow collection of data without human interaction, the analogy is drawn to a watchman's clock and key system. To track the movement of the watchman on his or her rounds, the watchman would carry a key and key in at each location to show progress through the rounds. As the inventive system is set up, stations include sensors to demonstrate the presence of a particular batch at the station, the sensors sense the presence of RFID tags. RFID tags can contain sufficient information to identify each asset in the system in turn and once identified and authenticated, the asset having the tag can log in to the system by means of a distinct network link. In this way, the network is not exposed to other signals not intended for or appropriately admitted to communicative connection with the master server 500. Most importantly, the batch with which each RFID tag is associated is uniquely identified with a serial number and provides an identity to which all sensed data collected may be associated.

Referring then to FIG. 2A, information relating to the serialized batch of concrete mix is handed off from the batch plant 110, to the mixer truck 120 in a load out interrogation and data exchange 115a at a time when the mixer truck 120 receives that particular batch of concrete mix from the batch plant 110. In this embodiment, much of the relevant data (though not necessarily all collected data) to follow the batch of concrete mix. In a similar manner, when this same batch of concrete mix is delivered to the concrete pumper 130, the mixer truck 120 and the concrete pumper 130 effect a delivery interrogation and data exchange 125a. Before embarking upon a discussion of the remaining transfer of data in and between the various stations, the means of data transfer is to be examined.

When planning and designing a data exchange system for tracking concrete production and placement, one must include a means to track each batch from the batching plant 110 to the final destination in either a construction form 150 or a test pour 160 and as the batch makes that transit, to identify all sensed conditions acquired in mixing, delivery, and placement of concrete mix and to make that association in a manner that uniquely associates these measured conditions with the relevant batch of concrete mix. Initially, the instant invention is configured to preserve the ability to automatically identify and track materials with no, or minimal human input, and to make transfer of this information readily and easily available to the system in an otherwise secure exchange. The invention further requires that the data exchange does not endanger workers involved in the mixing, delivery, and placement of concrete mix. Finally, the system must be secure such that data cannot be mistakenly attached to a wrong batch of concrete mix. The system must be relatively simple and durable enough to withstand the hostile construction environment. In some embodiments, the elements of the system should be simple enough to be added to conventional equipment with a minimum of modification thereto so that the significant capital expense of such as mixer trucks need not be duplicated to adopt the system.

Still a further consideration unique to liquid products of any sort is that any tag to identify the batch cannot be attached directly to the concrete mix while it is either a dry and as of yet unhydrated state or in a ready-mix liquid state. Due to plasticity of the concrete mix, tagging must be affixed to vessels rather than directly to the concrete mix. Because events such as receipt of the concrete mix issued from the batch plant 110 within the mixer truck 120 are time-stamped, a vessel such as the mixing drum of the mixer truck 120 may have a tag unique to the mixer truck 120 which may, however, carry multiple loads of concrete mix during a day. Each load is distinguished by the time when any one load resides within the mixer truck 120. So, some means of automatic identification system (“Auto-ID” system) is necessary to track individual loads without significant human intervention.

In the hostile construction environment having both water and abrasives, the RFID tag proves to be well-suited. RFID (radio frequency identification) is a wireless sensor technology, based on the detection of electromagnetic signals and radio frequencies, which are used to capture and transmit data from or to a tag. When triggered by an electromagnetic interrogation pulse from a nearby RFID reader device, the tag transmits digital data, usually an identifying inventory number, back to the reader. Every RFID system consists of three components: a scanning antenna, a transceiver and a transponder. When the scanning antenna and transceiver are combined, they are referred to as an RFID reader or interrogator. The RFID reader the inventive system comprises, as discussed below, is a network-connected device that can be portable or permanently attached to vehicles, equipment, and stations. It uses radio waves to transmit signals that activate the tag. Once activated, the tag sends a wave back to the antenna, where it is translated into data.

Without a doubt, conceptually, RFID and barcodes systems are relatively similar, where both are proposed to present the capability of quick and reliable technologies in item identification and tracking. However, the two technologies have different methods for reading and writing data. With RFID systems, the reader interrogates or scans the tag using Radio Frequency (RF) signals and does not need a direct line of sight between the reader and the tag. On the other hand, in the Barcode technology, a laser beam is used by the reader to scan a printed label and requires a direct line of sight between the optical scanner and the barcode labels. Barcodes use universal product codes (UPC), whereas RFID uses electronic product codes (EPC).

At the presently preferred embodiment, a conventional RFID tag is a portable memory device located on a chip that is encapsulated in a protective shell and can be embedded in any object which stores dynamic information about the object. Tags consist of a small integrated circuit chip, coupled with an antenna, to enable them to receive and respond to radio frequency queries from a reader. Tags have distinct memory configurations which can be categorized as Read-Only (RO), Write Once, Read Many (WORM), and Read-Write (RW) in which the volume capacity of their built-in memories varies from a few bits to thousands of bits. RFID tags can be classified into active tags (battery powered) and passive tags, which are powered solely by the electromagnetic field emanated from the reader, and, hence, have an unlimited lifetime.

Reading and writing ranges depend on operation frequency (low, high, ultra-high, and microwave):
LF (low frequency): frequencies between 125 and 134.2 kHz. The maximum detection range of a tag responding to this frequency is about 50 cm. The characteristics associated with this frequency range are: high price even with large volumes, low impact of a metallic or liquid environment on reading performance. There are several standards (ISO 18000-2, ISO 11784, ISO 14223, etc.) but the frequency ranges used are the same all over the world. This standard is used, for example, for animal tattooing or car keys.
HF (high frequency): frequency of 13.56 MHz. The maximum detection range of a tag responding to this frequency is about 1 m. The characteristics associated with this frequency are as follows: lower price than LF tags, suitable for applications requiring contact reading without a large volume of tags to be read, global frequency (the same in all countries). Examples of applications are access control or electronic passports. Note that NFC (near field communication) belongs to this category.
UHF (ultra-high frequency): frequencies between 864 and 928 MHz. The maximum detection range of a passive tag in this frequency range is approximately 3 to 20 m, depending on the propagation conditions. The characteristics associated with this frequency range are as follows: lower price than LF and HF tags for large volumes, suitable for applications requiring reading distance and a large volume of tags to be read very quickly, tags dedicated to constrained environments (metal, liquid, etc.). The uses of this standard are, for example, logistics, item identification, traceability, tolls.
SHF (super-high frequency): frequencies between 2.45 and 5.8 GHz. The maximum detection range of an active tag in this frequency range is about 100 m. The characteristics associated with this frequency range are as follows: relatively similar performance to UHF, high sensitivity to metal and liquid environments, directionality of tag detection. The frequency range in which SHF RFID systems operate are those which are globally unlicensed, allowing these systems to be used globally. However, these frequency bands are crowded and can be prone to interference as many devices such as cordless phones and microwave ovens use these frequencies.

A passive RFID tag, as its name indicates, is purely passive, i.e., it does not integrate either a battery or a radio frequency transmitter. A passive tag uses the wave (magnetic or electro-magnetic) from the interrogator (RFID reader+antenna) to power the embedded electronic circuit (i.e., its integrated circuit, IC, called the chip) and allows it to communicate the information contained in its memory by using the backscattering principle.

Access control systems are an important part of the security of government buildings, companies, schools, residences and private areas and RFID technology has been widely adopted in access control systems. These systems often use RFID identification cards based on the IEC/ISO 14443, IEC/ISO 15693, or IEC/ISO 18000 standards. The identification cards work much like a traditional key for unlocking doors or otherwise granting access. In the preferred embodiment of the instant invention, the RFID tag is used both as an identifying and a security token on each of the stations of FIGS. 1, 2, and 3, i.e. the batch plant 110; the mixer truck 120; the concrete pumper 130; the work site 140; and each of the construction form 150 and the test pour 160. It is by means of these RFID cards that the door to the local network each station comprises to accommodate the inventive data exchange. RFID cards only exchange identification data but it is that identification data that enables the network login in order facilitate the data relating to the pozzolanic reaction of a specific batch of concrete mix that will be, likewise, exchanged between a first station and a second station, e.g. from the batch plant 110 to the mixer truck 120.

The data exchange depicted in FIG. 2A and based on the security role of RFID is composed of three major components including an RFID system, a middleware system and a network system.
a) RFID system consists of readers, tags and antennas. Identification of target is done with a unique Electronic Product Code (EPC) saved in RFID tag. RFID tags are wireless devices which communicate with RFID readers. Readers include transport, receiver and microprocessor responsible for reading or writing tag information. Radio frequency signals between RFID tag and the reader are transmitted by antennas. This layer collects information and identifies the physical world especially as defined by the several stations: the batch plant 110; the mixer truck 120; the concrete pumper 130; the work site 140; and each of the construction form 150 and the test pour 160.
b) A middleware system is responsible for information transmission, initial processing of information, and classification of data. This layer includes key server and Object Naming Service (ONS) server.
c) The network system is responsible for analysis, processing, control, and decision making of information to implement customized services ordered by the job specification and controlling the connection between stations. In various of the embodiments, this layer includes network connection, where appropriate, Internet connection and a database (by way of nonlimiting example, a PML database). Exploiting the nonlimiting example—while the Electronic Product Code identifies individual products, all the useful information about the product would be written in a new, standard computer language called the Physical Markup Language (“PML”). The proposed subset of PML is based on the widely accepted extensible Markup Language (XML). It will provide a common method for describing physical objects and is broadly hierarchical. In this nonlimiting example, the PML is configured to include data that changes constantly (dynamic data) and data that changes over time (temporal data). Dynamic data in a PML file might include the temperature of a batch of concrete mix, number of turns of a drum of the mixer truck 12, or vibration levels from a concrete pumper 130. Temporal data changes discretely and intermittently throughout the concrete mix batch's life such as the temperature of the batch. One further example is any of locations of the batch which, except for the batch plant 110, change in transit. By making all this information available as associated with time stamps in a PML file, concrete companies will be able to use information in new and innovative ways.

Through the EPC code saved in RFID tag, the reader collects data from the tag. In the embodiment described in FIG. 2B, the middleware system can find the relevant information of the object by finding the corresponding IP address on the Object Naming Server on the Internet through this EPC code and the information is processed and managed by the middleware system. Relative to local connections between relevant stations, login information and dynamic host configuration protocol (“DHCP”). In the embodiment described in FIG. 2A, the DHCP allows devices to join an ad hoc mobile network without the need for any individual device setup. The DHCP protocol consists of two components:
A DHCP client; and
A DHCP server.
   A DHCP client is responsible for requesting an IP address and assigning it to the station and components thereon.

In the embodiment depicted at FIG. 2A, a central authority, likely the concrete company located at the batch plant 110, though not necessarily so, maintains a dynamic directory of public keys of all registered stations, i.e. the batch plant 110; the mixer truck 120; the concrete pumper 130; the work site 140; and each of the construction form 150 and the test pour 160. In addition, each registered station reliably knows a public key for the authority, with only the authority knowing the corresponding private key.

Using this dynamic directory also allows for spot checking any batch during each of the hand-offs of the batch from station to station. For example, if the batch is tested and that test yields a measurement corresponding to slump of the batch at the batch plant 110 just prior to loading a vessel on a mixer truck 120 and that same batch is tested on the mixer truck 120 upon loading, and where those slump readings vary widely relative to a predicted value, the system immediately “tags” the batch to indicate the nature of the issue and, if the reason for the change in slump can be detected and remedied, the batch can, possibly, be used once remedied. But the rapid change in slump is noted and the batch is isolated.

The following steps occur in a nonlimiting example of the authentication of the RFID as an authorized network participant:
1. The RFID reader, representing the respective registered station, sends a time stamped message to the key authority containing a request for the current public key of relevant station ad hoc station server.
2. The authority responds with a message that is encrypted using the authority's private key, PRauth. Thus, respective registered station's network element, optionally located on the reader, is thereby able to decrypt the message using authority's public key. Therefore, reader is assured that the message originated with the central authority. The message includes the following:
   i) Server's public key (PUb) which the reader can use to encrypt ad hoc network messages destined for server.
   ii) The original request used to enable the reader to match this response with corresponding earlier request and to verify that the original request was not altered before reception by the authority.
   iii) The original timestamp given so reader can determine that this is not an old message from the authority containing a key other than server's current public key.
3. Reader stores server's public key and also uses it to encrypt a message to server, containing an identifier of reader (IDr) and a first nounce N1 which is used to identify this transaction uniquely.
4. Server retrieves a reader's public key from the authority in a similar way.
5. Server sends a message to reader encrypted with and containing readers nounce N1 as well as a new nounce generated by the server N2, because only an authorized station server could have decrypted message based upon the key stored at (3) given the presence of N1 in the message (4) assures reader that the correspondent is the sender.
6. Reader returns N2, which is encrypted using server's public key to assure the server that the correspondent is a relevant station reader.

Referring, then, to FIG. 2A, the mixer truck 120 pulls up to the batch plant 110 to receive a batch of concrete mix. The RFID tag on the mixer truck 120 identifies to the batch plant 110 the particular mixer truck 120 to establish a secure ad hoc wireless network connection in the manner as described above. A broad range of wireless technologies and their devices have emerged and been developed based on Wireless Personal Area Networking-WPAN (e.g. Bluetooth), Wireless Local Area Networking-WLAN (e.g. Wi-Fi) and Wireless Wide Area Networking-WWAN (e.g. GSM). Bluetooth is a technology specification for low cost, short range wireless links between portable handheld devices, mobile phones and PCs, which acts through 2.4 GHz and covers a range of up to 10 m. While not the exclusive solution for station-to-station communication, the RFID's role can be fulfilled by a Bluetooth network (or any of the listed or like technologies). Either enables the exchange of sufficient login information thereby to allow the pairing of, for a nonlimiting example, the concrete mixer truck 220 and the concrete pumper 230 with no human intervention. Because all data exchange is between physically proximate stations, any near field connection technology such as Bluetooth will serve as a data exchange vehicle, the RFID tag serving to facilitate pairing.

By means of any of the envisioned networks, at a load out data exchange 115a, the batch plant 110 transmits all relevant PML data relating to that batch of concrete mix to the mixer truck 120, including, in the presently preferred embodiment, such data as the concrete temperature, number of mixing movements the batch received at the batch plant 110, temperatures, grades and specifications of each of the Portland cement, aggregate or aggregates used in mixing, time since initiating hydration, volume and mass of charge, concrete air content, measured slump, measured ambient humidity and temperature, and any instructions to the mixer truck 120 such as the GPS location of the work site 140, proposed route, and projected time of arrival, numbers of drum rotations and drum rotational speed to be performed in transit, projected pour time.

Having both the batch of concrete mix and relevant data describing that batch, the mixer truck 120 makes the transit to the location of the concrete pumper 130 (in this nonlimiting example, the concrete pumper 130 pumps the concrete into the construction form 150 from outside of the tightly-defined geographic limits of work site 140, though, the act of pumping results in the checking of the batch into the work site 140—other configurations are possible as noted above). Once at the location of the concrete pumper 130, a delivery data exchange 125a is effected.

Any information about the transit from the batch plant 110 to the concrete pumper 130, such as the number of turns and velocity of those turns of the drum that the mixer truck 120 exerted on the concrete mix, and at selected intervals, the ambient temperature and humidity, the route and the time consumed in transit, the temperature of the concrete in regular intervals between charging at the batch plant 110 to the pour at either of the construction form 150 or the test pour 160 or each. Thus, at this point, the concrete pumper 130 has the most current data relevant to that batch, that data transferred back to the batch plant 110 at the next load out data exchange 125a.

Now, because of its intimate proximity, in operation, to each of the work site 140, the construction form 150, and the test pour 160, the concrete pumper 130 is used as the reservoir for data at the time of the pour. The concrete pumper 130 enters the work site 140. At that time, in a site arrival data exchange 135a, the time of check-in at the site is noted as transmitted from the work site 140 as well as meteorological conditions there.

Two further data exchanges are based upon two distinct pours, that into the construction form 150 and that into the test pour 160, the data exchanges being the pour out data exchange 145 and the test pour data exchange 155, respectively. In each of the two types of pours, optionally, wireless maturity sensors (ASTM C1074) are placed to monitor and collect data as to each pour. These data, at least, are part of the makeup of each of the pour out data exchange 145 and the test pour data exchange. At the end of each pour, initial values are sent to the concrete pumper 130. In subsequent visits to the work site 140, the concrete pumper 130 will collect such additional data as may be available. The ad hoc mobile networks will present all available data in each visit, not merely those relative to the most recently delivered concrete batch.

Wireless maturity sensors are a relatively new development, but they work based on the principle that concrete strength is directly related to its hydration temperature. Wireless maturity sensors are placed on rebar before concrete is poured and stay in the concrete as it cures. Temperature data is collected by the sensors and sent to a station dedicated reader using a wireless connection. The compressive strength data is updated in real-time and is considered one of the most accurate and reliable measurements of concrete strength. Concrete strength is directly related to its hydration temperature history. Wireless sensors are placed within the concrete formwork, secured on the rebar, before pouring. Temperature data is collected by the sensor and uploaded to any smart device within an app using a wireless connection. This information is used to calculate the compressive strength of the in-situ concrete element based on the maturity equation that is set up in the app. Compressive strength data is given in real-time and updated, for example, every 15 minutes and entered with a time stamp. As a result, the data is considered more accurate and reliable as the sensors are embedded directly in the formwork, meaning they are subject to the same curing conditions as the in-situ concrete element. This also means no time is wasted onsite waiting for results from a third-party lab.

There are at least two conventional data networking options to enable embodiments of the invention for what is commonly known as the Internet of Things (IOT). Companies or developers in the process of building out an IoT deployment will often compare cellular vs. LoRaWAN for connectivity.

LoRaWAN is a low-power, wide area networking protocol built on top of the LoRa radio modulation technique. It wirelessly connects devices to the internet and manages communication between end-node devices and network gateways. Usage of LoRaWAN in industrial spaces and smart cities is growing because it is an affordable long-range, bi-directional communication protocol with very low power consumption—devices can run for ten years on a small battery. It uses the unlicensed ISM (Industrial, Scientific, Medical) radio bands for network deployments.

LoRaWAN is generally selected in commercial settings when the value of a single monitored device is relatively low, but the value of collecting data from hundreds of devices is high. Conventionally, LoRaWAN-based IoT solutions, are used by supply chain and logistics companies to successfully track high-value assets, including those in transit. Vehicles, goods, and other support are conveniently tracked over broad geographic regions and in severe circumstances because of the technology's remarkable range, low power consumption, and GPS-free localization. LoRa-based devices have a module that connects with a gateway, which is a local central location thereby generally locating the module geographically. The gateway acts as a conduit between the devices and the server.

Cellular is another good fit and is conventionally used for networking assets when those connected assets are of high value or because they're generating revenue during operation. In exchange for its greater subscription expense, a cellular IoT network connection provides end-to-end security. Modern mobile networks encrypt traffic to prevent attackers from accessing IoT-enabled devices and block network peers from inspecting the setup. One benefit is protection from eavesdropping, and that nobody else can tamper with the devices. In addition, customers who use cellular IoT don't need to worry about IoT breaches affecting their home or business networks, as hackers can't attack networks from a device connected only to cellular.

As stated above, any of the mentioned data exchanges, the load out data exchange 115a, the delivery data exchange 125a, the site arrival data exchange 135a, the pour out data exchange 145a, and the test pour data exchange 155a, are not limited to data about the most immediately recent batch of concrete mix then being delivered. As a nonlimiting example, wireless sensor data from either or each of the construction form 150 and test pour 160, will reflect all prior pours such that when a next site arrival data exchange 135a will be accompanied by all of the prior data collected in a pumper data exchange 135b so all concrete maturity data available and all meteorological data collected relative to all prior pours is conveyed to the concrete pumper 130. And when the mixer truck 120 is next in the proximity to the concrete pumper 130 and its RFID tag, all PML data from any source may be sent to the mixer truck 120 in a second delivery data exchange 125b. Then, too, all PML data in the possession of the mixer truck 120 is delivered to the batch plant 110 for analysis and machine learning, the analysis to include quality control review. Because of a full cycle among the stations, all relevant information will ultimately reside with the batch plant 110.

Progressing now to review of the second embodiment of the data exchange scenario, that depicted in FIG. 2B, data is collected from each of the stations, in this alternate embodiment, the batch plant 210; the mixer truck 220; the concrete pumper 230; the work site 240; and each of the construction form 250 and the test pour 260 and transmitted to a data center 270, “in the cloud.” This second embodiment also relies, in part, on the RFID card to enable network communication with the data center 270. The RFID tags will include an address which will be such as to uniquely identify each batch of concrete mix and any data relevant to the batch by virtue of time and location of each source of each data transmission.

In a presently preferred embodiment, a cellular modem is used to allow each of at least the batch plant 210; the mixer truck 220; the concrete pumper 230 and the work site 240 to communicate with the data center 270. The working of cellular networks includes significant sophistication in terms of antennas, hardware, radio waves, modulation, and many other areas.

Cellular modules are not new. A large industry has grown up around the technology for servicing the machine-to-machine (M2M) applications. For example, vending machines in remote locations are often connected to the company's computers via a cellular link. Given that designing a cellular modem from scratch is extremely difficult, the solution comes in the form of plug-and-play modules that are tested, verified, and certified.

Cellular module makers now offer products specifically targeted at the maker that are designed to be compatible with popular single board computers (“SBCs”) from suppliers such as Microchip Technology, Arduino, and Adafruit. Using a cellular module, a maker project SBC can send data directly to another remote Internet-connected device, such as the maker's smartphone (when they are away from the SBC) or a cloud server, without needing a network router gateway. Moreover, a cellular modem offers a range of up to tens of kilometers, extending the range of wireless maker projects beyond the home. Cellular modems also dispense with the need for inconvenient password entry to add the wireless device to a LAN.

Cellular communications use licensed frequencies. While there is a fee for use, the upside is they are tightly controlled and hence relatively free of the congestion and associated interference that can trouble a license free band, such as 2.4 GHz. To gain access to the network, the process is the same as that adopted by the mobile phone: users subscribe to a local carrier. They then obtain a subscriber identity module (SIM) that upon insertion authenticates the module, enabling a set amount of data upload and download according to the terms of the contract.

In addition to the license fees, there are some other drawbacks to cellular connectivity. The modules are relatively large, heavy, expensive, and power consumption is much greater than Bluetooth low energy, LoRaWan, or even by Wi-Fi. Moreover, connecting directly to the cellular network is more involved than connecting to a smartphone or router.

In addition, there are several cellular technologies (for example, GSM, GPRS, and CDMA), several generations of each technology in commercial use (2G, 2.5G, 3G, 4G and 5G), and dozens of cellular bands across the globe. As a result, selecting a cellular module for a specific location will dictate the exact selection of a specific modem.

In an alternate embodiment, each of the stations, the batch plant 110; the mixer truck 120; the concrete pumper 130; the work site 140; and, in some embodiments, each of the construction form 150 and the test pour 160, include a mobile hotspot. A mobile hotspot is an ad hoc wireless access point that is created by a dedicated hardware device or a smartphone feature that shares the phone's cellular data. Other nearby devices can then use the shared hotspot to connect to the Internet.

Mobile hotspots are also known as portable hotspots. The hardware devices used to create them, officially known as pocket or travel routers, are sometimes referred to as mobile hotspots as well. They are also often generically known as Mi-Fis, although that name is owned by Novatel in the United States and many other countries.

Pocket routers access cellular signals and convert the cellular signals to Wi-Fi and vice versa, creating mobile Wi-Fi networks that can be shared by multiple users within about 10 meters of the device. In the presently preferred embodiment of the mobile hotspot infrastructure, the RFID tag provides an encrypted login password, enabling the network upon scanning the RFID tag. Still further, the RFID might, optionally, be assigned an IPV6 identity by which to communicate with the data center 270 and to identify itself to the data center 270 to enable batch-specific recording of attributes of the batch.

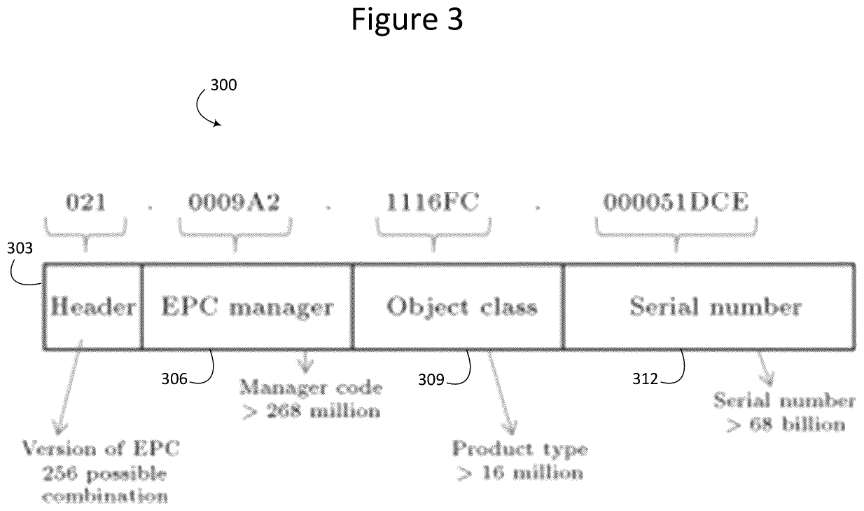

By associating a passive RFID tag such as a key card with a globally unique IPv6 address, this second embodiment uses access control and security policy mechanisms with Internet technologies to provide the desired access control and batch identification. Because moving objects can easily carry RFID tags, it is the time-stamped, station-identified event of reading the RFID tag at a particular station. Electronic Product Code (“EPC”) is a universal identifier that aims to render a unique identity to every possible physical object of the world. Referring, then, to FIG. 3, an EPC is a 96-bit number the RFID tag broadcasts to identify a particular tag amongst other tags. It distinguishes two identical products, and also provides the product's manufacture date, origin or batch number.

Referring to FIG. 3, the EPC 300, includes four identified segments:
Header 303—Designates a specific format of EPC with an 8-bit number that shows the length, type, structure, version, and partitioning scheme.
EPC Manager Number 306—A 28-bit number that indicates company manufacturer, i.e. indicator of ownership of the set of numbers, and uniquely identifies the company or company entity.
Object Class 309—A 24-bit number that identifies product types and is similar to a SKU (pronounced “skew”), short for stock keeping unit.
Serial Number 312—A 36-bit unique number that identifies each product instance, in the instant case, each batch of concrete.

Because each batch of concrete mix is assigned a unique serial number, compiling data relative to a batch considering the appropriate EPC 300 is an exercise to populate a conventional database. With each scan or swipe, at any of the stations, a location (station-wise) and a time are associated with the batch. Any sensed data drawn from any of the above-described sensors may be appended to the data to give a fuller picture of the state of a batch of concrete for further analysis and optimization. Where a global positioning service (“GPS”) or other localization protocol and mechanism is used, the timestamping can be augmented with position stamping to state the geographic position as well as the station-wise positioning of concrete in the series of stations set out in FIGS. 1, 2A, and 2B.

When the tag is swiped at the reader, the application host creates an IPV6 address by combining the network prefix configured at the reader with the tag's identity. While there are several methods proposed for standardization among the regulatory bodies, one example will suffice to explain this virtualization. As is noted in FIG. 3, of the segments of the EPC 300, the serial number segment 312 is exploited. To implement a virtualization of the RFID tags, a middleware residing on the reader will make the address assignments under IPV6. First, the reader will read the RFID tag identifier, and then send it to the RFID host (HR). The latter is an application linked to the reader; it is responsible for converting the tag identifier into an IPV6 address. It also allows the storage of tag identifiers in a database. Then, it sends an RS message (Router Solicitation) to the router which provides it with the Netid identifier (the network prefix) to generate an IPV6 address. The HR generates a unique IPV6 address interface identifier according to the conventional algorithm and adds it to the network ID received from the router to generate the IPV6 address of the RFID tag.

RFID tags can, in some embodiments, have a saved EPC code on their chip. The antenna on the reader then transfers this code to the reader. The reader reads the code and transfers to the Object Name System (ONS), which eventually can obtain precise information about the place of objects. However, to achieve information in a real-time fashion, the connection between the object should be established through the Internet by IPv6.

RFID tags cannot directly use IPV6. Thus, IPv6 is defined in different ways for the tag. Tag Reader has an ID which distinguishes the tagged object from other objects. Thus, ID can be used for creating and allocating an IPV6 address. The tag identity together with the constructed IPV6 address and a timestamp are stored on the database. An EPC with the length of 64 bits maps well in the IPV6 address format and can result in globally unique addresses. A key benefit of the proposed solution is that there is no need to change the design of existing RFID technology with its EPC namespace conventions. The application can be installed on a computer connected to the reader or, in the preferred embodiment, within the microcontroller 411, and then all objects with RFID tags that pass this reader will put the objects online and thereby giving them the ability to communicate over the Internet if the tag is within range of a reader.

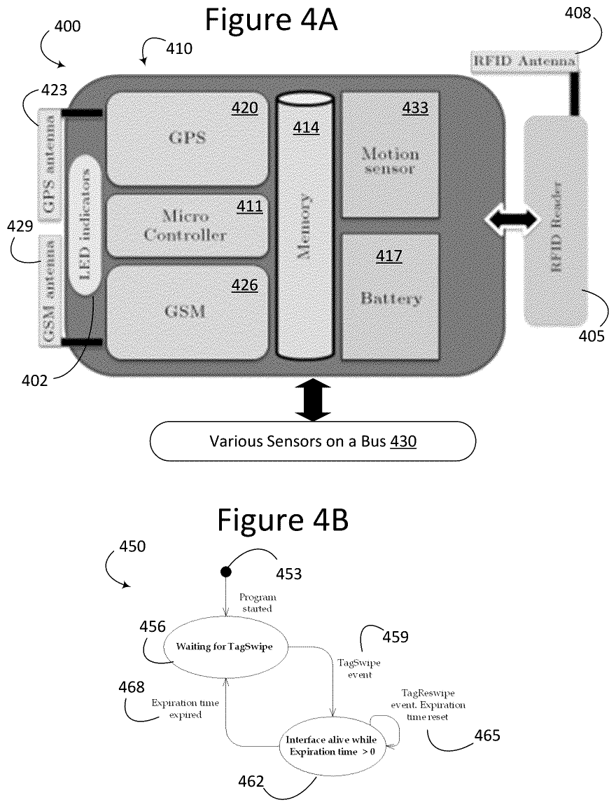

Referring to FIG. 4A, an exemplary RFID reader assembly 400, includes an RFID antenna 408 with an RFID reader 405 that interprets backscatter from an RFID tag to translate the same into discrete numeric messages and a computing structure 410 including a microcontroller 411 with a memory 414 which includes each of RAM and ROM, and is powered, as is the whole of RFID reader assembly 400 by, optionally, a battery 417 (or by any analogous power source which might serve to supply such electrical power) though that both self-locates with a global positioning system (“GPS”) antenna 423 and its GPS receiver 420, and then communicates with the network using any of the networked solutions discussed above and grouped collectively as Global System for Mobile Communications (GSM) including a GSM antenna 429 with its allocated receiver, a GSM receiver 426. Importantly, the microcontroller 411 communicates with any of the relevant sensors as the same are connected on a data bus 430. It is by this means that data can be collected and queued for transmission, ultimately, to the data center 270 or to the batch plant 110, where all of the data is used for machine learning at a computer master server 500. The microcontroller 411 coordinates the taking of sensed data from the various sensors, associates that data with the relevant batch; time-stamps the data, and stores the same in memory 414. Thus, the microcontroller 411 facilitates movement of data, suitably identified, to the master server 500.

While the envisioned reader is generally to function autonomously without human intervention, the option of a dashboard of status light emitting diodes (“LED indicators”) is presented to allow the troubleshooting or status reporting by indicating, for example, one for the fact of having resolution of a GPS position and second for a GSM connection (to indicate a viable GSM connection); RFID detection indicator LED (to indicate presence of an RFID tag within the detection scope of the reader), and, of course, one to indicate power being supplied to the unit. One can readily understand that in installation or maintenance of the RFID reader assembly 400, knowing that the various elements are in working order is very useful. Such LED indicators have proven to be extremely useful in analogous applications such as in the case of wireless routers used for local area networks (LANs) to indicate connections between the router and a wide area network modem (WAN). Further, distinct LED indicators are employed to show establishment of the wireless network as well as any wired connections to the network.

Much of the performance and enablement relative to compiling data from sensors and from sources of known data such as weather data and nature and source of cement is as described above. Also as referred to above, these readers reside at the identified stations, at least the batch plant 210 and the work site 240 (both of FIG. 2B), to read RFID tags on such as the mixer truck 220, the concrete pumper 230, and each of the construction form 250 and the test pour 260 would be used to establish network handshakes to move data to the data center 270. The same is true of the embodiment depicted in FIG. 2A where data moves from station to station therein.

The microcontroller 411 employs the RFID reader 405 through the RFID antenna 408 to establish that handshake enabling GSM connection by supplying protocols as a part of the RFID tag information. But, before this information is available to the RFID reader assembly 400 to establish the GSM connection, the RFID reader 405 must detect the presence of an RFID tag. FIG. 4B depicts a state diagram 450 which both assures the security of the network and conserves the power the RFID reader assembly 400. After bootup 453, in the first state, i.e. the waiting state 456, the RFID receiver 405 awaits receipt of a signal at the RFID antenna 408, a “TagSwipe” (as a nonlimiting example of a variable name) event to occur. Subsequently, when that TagSwipe event 459 is detected, the machine state advances to switch on each of the GPS receiver 420 and the network interface, i.e. the GSM receiver 426 and its antenna 429.

As described above, the network is put online and from the RFID EPC, the IPV6 address is constructed at a connection standby step 462, and it is kept alive as long the expiration time is greater than 0 (zero) seconds. A designated expiration time is selected to enable full transfer of all data each of the stations might hold. Tags and their respective stations' network are only reachable while they lie within reader range. When a tag's attachment to the network is virtualized, it is possible to set up an expiration value. This value effectively serves as the time the tag's virtual representation on the network can be reached. When a tag moves from one reader to another, the network prefix will change but the host suffix/interface ID will still match the tag's EPC. The tag will, in effect, change its network address every time it passes a new reader. Because the serial number of the batch remains the same, however, the system continues to associate the sensed data with the respective batch of concrete mix.

Fortunately, given that there is no need to have global coverage by a single batch plant 210 because the concrete mix will only remain plastic over relatively short distances in travel, the care-of address refers to the subnet of the RFID reader, where the tag is currently present. Whilst the care-of address is a globally unique address at the station where the reader is present, assigned to the host, i.e., the tag visiting a second station network will be part of those networks run by the concrete company so that the home agent address is specific to the concrete company using the issued tags.

In an alternate approach, e.g. where concrete mixer trucks 220 and concrete pumpers 230 are owned by independent vendors, mobility support can be obtained in a more distributed way by separating location and identity information.

In order to determine frames in which an RFID reader assembly 400 is sensitive to reading any RFID tags within the range of the RFID reader 405 working through its RFID antenna 408, a motion sensor 433 defines intervals of sensitivity in that periods of motion are not suitable for reading RFID tags. Referring, again, to FIG. 4A, the motion sensor 433 is included to selectively implement aspects of the RFID reader assembly 400 such as using the GPS receiver 420 to track movement of batch from one position to another and to turn off the RFID reader as no transfer of a batch of concrete mix can occur from or to a station while underway.

The optional motion sensor 433 is embodied as a three-axis accelerometer exploiting MEMS technology in the preferred embodiment. MEMS stands for microelectromechanical system and applies to any sensor manufactured using microelectronic fabrication techniques. These techniques create mechanical sensing structures of microscopic size, typically on silicon. When coupled with microelectronic circuits, MEMS sensors can be used to measure physical parameters such as acceleration. In the presently preferred embodiment, a variable capacitive MEMS three-axis accelerometer is used to sense movement of the RFID reader assembly 400.

Variable capacitive MEMS accelerometers are lower range, high sensitivity devices used for structural monitoring and constant acceleration measurements as the accelerometer 135. In contrast, piezoresistive MEMS accelerometers are higher range, low sensitivity devices used in shock and blast applications. For that reason, the presently preferred embodiment comprises a three-axis accelerometer 135 which sends a signal of movement of the RFID reader assembly 400.

To understand the utility of the three-axis MEMS accelerometer, which, in the presently preferred embodiment, comprises a micro-machined proof mass that is suspended between two parallel plates, it is useful to understand its operation. In such an exemplary accelerometer at least one mass is suspended on flexures that are attached to a ring frame. This configuration forms two air gap capacitors between the proof mass and upper and lower plates. As the proof mass moves when acceleration is applied, one air gap decreases, and the other gap increases, thereby creating a change in capacitance proportional to acceleration. The MEMS accelerometer includes a hermetic enclosure containing the proof mass and provides mechanical isolation and protection of the sensing mechanism. As such, MEMS accelerometers are well-suited for sensing in environments that are hostile to any non-solid-state precision electronic device. In an exemplary embodiment, the preferred embodiment is one in which ruggedness is enhanced using mechanical stops on the two outer wafers to restrict the travel of the proof mass.

Machine learning builds methods that ‘learn’, that is, methods that leverage data to improve performance on some set of tasks. Machine learning algorithms build a model based on sample data, known as training data, to make predictions or decisions without being explicitly programmed to do so. Learning algorithms work on the basis that strategies, algorithms, and inferences that worked well in the past are likely to continue working well in the future. These inferences can be obvious, such as “since the sun rose every morning for the last 10,000 days, it will probably rise tomorrow morning as well”. The learning system of a machine learning algorithm into three main parts.
A Decision Process: In general, machine learning algorithms are used to make a prediction or classification. Based on some input data, which can be labeled or unlabeled, the concrete batch algorithm will produce an estimate about a pattern in the data.
An Error Function: An error function evaluates the prediction of the model. If there are known examples, an error function can make a comparison to assess the accuracy of the model. In the instant case, predicting a quality of formed concrete under given conditions provides an ideal concrete batch, known as a model estimate. The actual quality of a batch of formed concrete is the real or what is known as known example. The difference between the real and the ideal is the error function.
A Model Optimization Process: If the model can fit better to the data points in the training set, then weights are adjusted to reduce the discrepancy between the known example and the model estimate thereby to minimize the error function. The algorithm will repeat this “evaluate and optimize” process, updating weights autonomously until a threshold of accuracy has been met.

In using the concrete batch data, the invention includes a branch of machine learning known as supervised learning, also known as supervised machine learning. It is defined by its use of labeled datasets to train algorithms that to classify data or predict outcomes accurately. As input data is fed into the model, it adjusts its weights until the model has been fitted appropriately, which occurs as part of the cross-validation process. Supervised learning helps organizations solve for a variety of real-world problems at scale, such as classifying spam in a separate folder from your inbox.

Supervised learning uses a training set to teach models to yield the desired output. This training dataset includes inputs and correct outputs, which allow the model to learn over time. The algorithm measures its accuracy through the loss function, adjusting until the error has been sufficiently minimized.

To achieve this machine learning, there is, necessarily, a means of scoring outcomes. In this aspect, in the context of concrete batching, evaluating various conditions and their impact upon the final quality of the resulting concrete. To explore these relationships using machine learning, naturally requires a machine. In the two alternate embodiments depicted in FIGS. 2A and 2B, all the collected batch data relating to any actions performed upon or conditions sense relative to any batch of concrete as collected by the depicted embodiments. All of this must be collected in a master server.

In one embodiment, the master server provides access to all the collected data and can allow tracking of any individual concrete batch. Still further, this master server can evaluate any sub-sample of training data to employ machine learning technology to optimize the quality of batches of concrete mix as delivered to construction forms 150, 250.

Certain embodiments are described herein as including logic or several components, modules, or mechanisms. Modules may constitute either software modules (e.g., code embodied on a machine-readable medium) or hardware modules. A “hardware module” is a tangible unit capable of performing certain operations and may be configured or arranged in a certain physical manner. In various example embodiments, one or more computer systems (e.g., a standalone computer system, a client computer system, or a server computer system) or one or more hardware modules of a computer system (e.g., a processor or a group of processors) may be configured by software (e.g., an application or application portion) as a hardware module that operates to perform certain operations as described herein.

In some embodiments, a hardware module may be implemented mechanically, electronically, or any suitable combination thereof. For example, a hardware module may include dedicated circuitry or logic that is permanently configured to perform certain operations. For example, a hardware module may be a special-purpose processor, such as a field-programmable gate array (FPGA) or an application specific integrated circuit (ASIC). A hardware module may also include programmable logic or circuitry that is temporarily configured by software to perform certain operations. For example, a hardware module may include software executed by a general-purpose processor or other programmable processor. Once configured by such software, hardware modules become specific machines (or specific components of a machine) uniquely tailored to perform the configured functions and are no longer general-purpose processors. It will be appreciated that the decision to implement a hardware module mechanically, in dedicated and permanently configured circuitry, or in temporarily configured circuitry (e.g., configured by software) may be driven by cost and time considerations.

Accordingly, the phrase “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where a hardware module comprises a general-purpose processor configured by software to become a special-purpose processor, the general-purpose processor may be configured as respectively different special-purpose processors (e.g., comprising different hardware modules) at different times. Software accordingly configures a particular processor or processors, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple hardware modules exist contemporaneously, communications may be achieved through signal transmission (e.g., over appropriate circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, later, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module implemented using one or more processors.

Similarly, the methods described herein may be at least partially processor-implemented, with a particular processor or processors being an example of hardware. For example, at least some of the operations of a method may be performed by one or more processors or processor-implemented modules. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)).

The performance of certain of the operations may be distributed among the processors, not only residing within a single machine, but deployed across several machines. In some example embodiments, the processors or processor-implemented modules may be in a single geographic location (e.g., within a home environment, an office environment, or a server farm). In other example embodiments, the processors or processor-implemented modules may be distributed across several geographic locations.

The modules, methods, applications and so forth described in conjunction with FIGS. 1-4B are implemented in some embodiments in the context of a machine and an associated software architecture. The sections below describe a representative architecture that is suitable for use with the disclosed embodiments.

Software architectures are used in conjunction with hardware architectures to create devices and machines tailored to specific purposes. For example, a particular hardware architecture coupled with a particular software architecture will create a mobile device, such as a mobile phone, tablet device, or so forth. A slightly different hardware and software architecture may yield a smart device for use in the “Internet of things” or IoT while, yet another combination produces a server computer for use within a cloud computing architecture. Not all combinations of such software and hardware architectures are presented here as those of skill in the art can readily understand how to implement the inventive subject matter in different contexts from the disclosure contained herein.

FIG. 5 is a block diagram illustrating components of a machine 500, according to some example embodiments, able to read instructions from a machine-readable medium (e.g., a machine-readable storage medium) and perform any one or more of the methodologies discussed herein. Specifically, FIG. 5 shows a diagrammatic representation of the machine 500 in the example form of a computer system, within which instructions 516 (e.g., software, a program, an application, an applet, an app, or other executable code) for causing the machine 500 to perform any one or more of the methodologies discussed herein may be executed. For example, the instructions 516 may cause the machine 500 to execute routines to analyze data accumulated by the system to perform machine learning thereon. The instructions 516 transform the general, non-programmed machine 500 into a particular machine 500 programmed to carry out the described and illustrated functions in the manner described. In alternative embodiments, the machine 500 operates as a standalone device or may be coupled (e.g., networked) to other machines.

In a networked deployment, the machine 500 may operate in the capacity of a server machine or a client machine in a server-client network environment, or as a peer machine in a peer-to-peer (or distributed) network environment. The machine 500 may comprise, but not be limited to, a server computer, a client computer, a personal computer (PC), a tablet computer, a laptop computer, a smart telephone, or any machine capable of executing the instructions 516, sequentially or otherwise, that specify actions to be taken by machine 500. Further, while only a single machine 500 is illustrated, the term “machine” shall also be taken to include a collection of machines 500 that individually or jointly execute the instructions 516 to perform any one or more of the methodologies discussed herein.

The machine 500 may include processors 510, memory/storage 530, and I/O components 550, which may be configured to communicate with each other such as via a bus 502. In an example embodiment, the processors 510 (e.g., a central processing unit (CPU), a reduced instruction set computing (RISC) processor, a complex instruction set computing (CISC) processor, a graphics processing unit (GPU), a digital signal processor (DSP), an application specific integrated circuit (ASIC), a radio-frequency integrated circuit (RFIC), another processor, or any suitable combination thereof) may include, for example, processor 512 and processor 514 that may execute the instructions 516.

The term “processor” is intended to include multi-core processor that may comprise two or more independent processors (sometimes referred to as “cores”) that may execute instructions 516 contemporaneously. Although FIG. 5 shows multiple processors 510, the machine 500 may include a single processor with a single core, a single processor with multiple cores (e.g., a multi-core process), multiple processors with a single core, multiple processors with multiples cores, or any combination thereof.

The memory/storage 530 may include a memory 532, such as a main memory, or other memory storage, and a storage unit 536, both accessible to the processors 510 such as via the bus 502. The storage unit 536 and memory 532 store the instructions 516 embodying any one or more of the methodologies or functions described herein. The instructions 516 may also reside, completely or partially, within the memory 532, within the storage unit 536, within at least one of the processors 510 (e.g., within the processor's cache memory), or any suitable combination thereof, during execution thereof by the machine 500. Accordingly, the memory 532, the storage unit 536, and the memory of processors 510 are examples of machine-readable media.

As used herein, “machine-readable medium” means a hardware device able to store instructions 516 and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable medium” should be taken to include a single physical medium or multiple physical media (e.g., a centralized or distributed database, or associated caches and servers) able to store instructions 516.

The term “machine-readable medium” shall also be taken to include any physical medium, or combination of multiple physical media, that is capable of storing instructions (e.g., instructions 516) for execution by a machine (e.g., machine 500), such that the instructions, when executed by one or more processors of the machine 500 (e.g., processors 510), cause the machine 500 to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

The I/O components 550 may include a wide variety of components to receive input, provide output, produce output, transmit information, exchange information, capture measurements, and so on. The specific I/O components 550 that are included in a particular machine will depend on the type of machine. For example, portable machines such as mobile phones will likely include a touch input device or other such input mechanisms, while a headless server machine will likely not include such a touch input device. It will be appreciated that the I/O components 550 may include many other components that are not shown in FIG. 5.

The I/O components 550 are grouped according to functionality merely for simplifying the following discussion and the grouping is in no way limiting. In various example embodiments, the I/O components 550 may include output components 552 and input components 554. The output components 552 may include visual components (e.g., a display such as a plasma display panel (PDP), a light emitting diode (LED) display, a liquid crystal display (LCD), a projector, or an organic light emitting diode (OLED) display, acoustic components (e.g., speakers), haptic components (e.g., a vibratory motor, resistance mechanisms), other signal generators, and so forth. The input components 554 may include alphanumeric input components (e.g., a keyboard, a touch screen configured to receive alphanumeric input, a photo-optical keyboard, or other alphanumeric input components), point based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further example embodiments, the I/O components 550 may include biometric components 556, motion components 558, environmental components 560, or position components 562, among a wide array of other components. For example, the biometric components 556 may include components to detect expressions (e.g., hand expressions, facial expressions, vocal expressions, body gestures, or eye tracking), measure bio signals (e.g., blood pressure, heart rate, body temperature, perspiration, or brain waves), identify a person (e.g., voice identification, retinal identification, facial identification, fingerprint identification, or electroencephalogram based identification), and the like. The motion components 558 may include acceleration sensor components (e.g., accelerometer), gravitation sensor components, rotation sensor components (e.g., gyroscope), and so forth. The environmental components 560 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 562 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 550 may include communication components 564 operable to couple the machine 500 to a network 580 or devices 570 via coupling 582 and coupling 572 respectively. For example, the communication components 564 may include a network interface component or other suitable device to interface with the network 580. In further examples, communication components 564 may include wired communication components, wireless communication components, cellular communication components, near field communication (NFC) components, Bluetooth® components (e.g., Bluetooth® Low Energy), Wi-Fi® components, and other communication components to provide communication via other modalities. The devices 570 may be another machine or any of a wide variety of peripheral devices (e.g., a peripheral device coupled via a universal serial bus (USB)).

Moreover, the communication components 564 may detect identifiers or include components operable to detect identifiers. For example, the communication components 564 may include radio frequency identification (RFID) tag reader components, NFC smart tag detection components, optical reader components (e.g., an optical sensor to detect one-dimensional bar codes such as universal product code (UPC) bar code, multi-dimensional bar codes such as quick response (QR) code, Aztec code, Data Matrix, Dataglyph, MaxiCode, PDF416, Ultra Code, UCC RSS-2D bar code, and other optical codes), or acoustic detection components (e.g., microphones to identify tagged audio signals). In addition, a variety of information may be derived via the communication components 564, such as location via Internet Protocol (IP) geo-location, location via Wi-Fi® signal triangulation, location by detecting a NFC beacon signal that may indicate a particular location, and so forth.

In various example embodiments, one or more portions of the network 580 may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the public switched telephone network (PSTN), a plain old telephone service (POTS) network, a cellular telephone network, a wireless network, a Wi-Fi® network, another type of network, or a combination of two or more such networks. For example, the network 580 or a portion of the network 580 may include a wireless or cellular network and the coupling 582 may be a code division multiple access (CDMA) connection, a GSM communications connection, or other type of cellular or wireless coupling. In this example, the coupling 582 may implement any of a variety of types of data transfer technology, such as single carrier radio transmission technology (1×RTT), evolution-data optimized (EVDO) technology, general packet radio service (GPRS) technology, enhanced data rates for GSM evolution (EDGE) technology, third Generation Partnership Project (3GPP) including 3G. 4G, and 5G networks, universal mobile telecommunications system (UMTS), high speed packet access (HSPA), worldwide interoperability for microwave access (WiMAX), long term evolution (LTE) standard, others defined by various standard setting organizations, other long range protocols, or other data transfer technology.

The instructions 516 may be transmitted or received over the network 580 using a transmission medium via a network interface device (e.g., a network interface component included in the communication components 564) and utilizing any one of several well-known transfer protocols (e.g., hypertext transfer protocol (HTTP)). Similarly, the instructions 516 may be transmitted or received using a transmission medium via the coupling 572 (e.g., a peer-to-peer coupling) to devices 570. The term “transmission medium” shall be taken to include any intangible medium that can store, encode, or carry out instructions 516 for execution by the machine 500, and includes digital or analog communications signals or other intangible medium to facilitate communication of such software.

Having, now, described the hardware and software environment necessary to enable machine learning, it becomes necessary to grade each concrete batch for its resulting qualities. Also, as described above, there is an interface for the user to enter data relative to the designated batch, thereby scoring that batch for its requisite construction qualities. For such scoring there is no reason to require novel solutions for scoring. There exist several important procedures to derive scoring procedures as results. The concept here is not that all tests are necessary. Rather, there exist several tests that are already defined, some of which are required by local or national building codes or in conformity with specific building permits. In the inventive model, any test taken can be entered through the master server or by a remote computer networked into the same master server described above relative to FIG. 5. Testing performed in conformity with the relevant standards give an opportunity for scoring that batch of concrete. Among the many tests that are probative as to concrete quality are:
ASTM C172 Sampling Freshly Mixed Concrete. ASTM C172 outlines the best practices for sampling ready-mix concrete. The biggest rule of concrete sampling is that samples should be taken from the ready-mix truck only after 10% of the concrete has been discharged and before 90% of the mix has been discharged. Samples should then be covered to protect against rapid evaporation.
ASTM C1064 Temperature of Freshly Mixed Concrete. Temperature measurements should be taken within 5 minutes of securing the concrete samples as per ASTM C1064. To get accurate readings, the thermometer needs to be placed in enough concrete that it is surrounded by at least three inches on all sides. The thermometer should remain inserted in the concrete for 2 minutes while you conduct other tests on the concrete.
ASTM C143 Slump of Hydraulic-Cement Concrete. Slump is a measure of consistency, or relative ability of the concrete to flow. If the concrete can't flow because the consistency or slump is too low, there are potential problems with proper consolidation. If the concrete won't stop flowing because the slump is too high, there are potential problems with mortar loss through the formwork, excessive formwork pressure, finishing delays and segregation. The concrete slump test is conducted by filling a slump testing cone with 12 inches of concrete and compacting the concrete inside with a ⅝ inch rod. Then the cone is raised releasing the concrete. You measure the amount the concrete slumps by taking the difference between the original 12-inch height and the height of the settled shape the concrete takes after releasing the concrete from the slump testing cone.
ASTM C231 Air Content of Fresh Concrete by the Pressure Method. Air content measures the total air content in a sample of fresh concrete but does not indicate what the final in-place air content will be, because a certain amount of air is lost in transportation, consolidation, placement and finishing. Three field tests are widely specified: the pressure meter and volumetric method are ASTM standards and the Chace Indicator is an AASHTO procedure. Concrete naturally contains some air voids just from the batching process, typically 1% or 2%. Using an admixture to entrain additional air is a practice that ensures resistance to freeze-thaw cycles through the development of a system of air bubbles. These voids allow forces from the expansion and contraction of water and solids to dissipate without damaging the concrete. Concrete Air Meters yield fast and accurate results using a method that is easy to run in the field. After proper placement of concrete in the bowl of the meter, air content is measured by pressurizing the upper chamber and releasing the volume of air into the lower chamber.
ASTM C173 Air Content of Fresh Concrete by the Volumetric Method (Roll-o-meter).
ASTM C138 Density (Unit Weight), Yield and Air Content of Concrete. Unit weight measures the weight of a known volume of the fresh concrete.
ASTM C31 Making and Curing Concrete Test Specimens in the Field. Compressive strength is the ability of material or structure to carry the loads on its surface without any crack or deflection. Compressive strength is tested by pouring cubes of fresh concrete and measuring the force needed to break the concrete cubes at prescribed interval as they harden. According to Building Code Requirements for reinforced concrete (ACI 318), if no single test more than 500 psi below the design strength and the average of three consecutive tests equal or exceed the design strength then the concrete is acceptable.
Compressive Strength of Concrete—Rebound Hammer or Schmidt Hammer (ASTM C805). A spring release mechanism is used to activate a hammer which impacts a plunger to drive into the surface of the concrete. The rebound distance from the hammer to the surface of the concrete is given a value from 10 to 100. This measurement is then correlated to the concrete's strength.
Penetration Resistance Test (ASTM C803). To complete a penetration resistance test, a device drives a small pin or probe into the surface of the concrete. The force used to penetrate the surface, and the depth of the hole, is correlated to the strength of the in-place concrete.
Ultrasonic Pulse Velocity (ASTM C597). Ultrasonic pulse velocity tests the velocity of vibrations through a concrete slab. The velocity of the vibrations gives data on the concrete's elasticity, density, and resistance to stress. The data can then be used to determine the overall strength of the concrete. Cons: This technique is highly influenced by the presence of reinforcements, aggregates, and moisture in the concrete element. It also requires calibration with multiple samples for accurate testing.
Pullout Test (ASTM C900). The pullout test uses a cast-in-place or post-cast rod with a head at the end. The rod is then pulled out using a special instrument that measures the amount of force required to pull the rod out of the concrete, which can then determine the compressive strength of the concrete. This test requires pullout test rods to be placed in multiple locations to get accurate readings from different areas in the concrete slab.
Drilled Core (ASTM C42). A core drill is used to extract hardened concrete from the slab. These samples are then compressed in a machine to monitor the strength of the in-situ concrete.
Cast-in-place Cylinders (ASTM C873). Test cylinders are cast to verify that the specified compressive strength of a concrete slab or structure is achieved. Typically, the cylinders are molded using a plastic tube that is 6 inches in diameter and 12 inches tall.

Understanding that consistent with the collection and exchange of data set out in the embodiments depicted in FIGS. 2A and 2B, each of the sensed attributes as described relative to FIG. 1, as discussed above include but are not limited only to those set out in FIG. 1, one rapidly understands these to be the various independent variables which drive the quality of the concrete batch to which those attributes apply. Such is the domain upon which the machine learning will apply. The master server will continue, on an iterative basis, to refine the role of each sensed attribute and will exercise its control over those actions to be performed upon the batch of concrete to optimize the qualities of the resulting concrete pour.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in enough detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

1. A method for mixing and for placing a batch of concrete mix in forms, the method comprising:
assigning a unique serial number to the batch of concrete, that serial number being stored in a database;
admitting a measured quantity of each of the concrete ingredients into a mixing vessel, the ingredients comprising:
a cement quantity of cementitious material;
a water quantity of water;
a sand quantity of sand; and
an aggregate quantity of aggregate; and
storing each of the cement quantity, the water quantity, the sand quantity and the aggregate quantity as stored attributes in association with the serial number;
curing the batch of concrete mix;
testing the cured batch of concrete mix to derive at least one performance criterion; and
storing the performance criterion in association with the serial number.

2. The method of claim 1, wherein the batch includes a plurality of batches and further comprising:
selecting a machine-learning algorithm to optimize the at least one performance criterion;
invoking the machine-learning algorithm to train a neural network model with the at least one performance criterion to generate a group of performance criterion data;
analyzing the neural network model produced by training for an accuracy; and
improving the accuracy by iteratively repeating the training of the neural network model by performing the method of claim 1 until a defined constraint is met.

3. The method of claim 2 wherein the machine-learning algorithm is selected from a database including a plurality of machine learning algorithms.

4. The method of claim 2 wherein the invoking trains the neural network model with the machine-learning algorithm of a first portion of the performance criterion data; and
wherein the analyzing analyzes the accuracy based on a second portion of the performance criterion data.

5. The method of claim 2 wherein the machine-learning algorithm is invoked in a stateless manner to train the neural network model.

6. The method of claim 2 wherein a plurality of machine-learning algorithms are stored in a plug-in model wherein each machine-learning algorithm can be dynamically edited, added or deleted.

7. The method of claim 3 wherein the selected machine-learning algorithm comprises a combination of the plurality of machine-learning algorithms in the database.

8. The method of claim 6 wherein the selected machine-learning algorithm comprises a combination of the plurality of machine-learning algorithms in the plug-in model.

9. The method of claim 1 wherein the stored attributes include additional attributes selected from an additional attributes group consisting of:
a measured temperature of the batch when the batch is in the mixing vessel;
a measured slump of the batch when the batch is mixed in the mixing vessel;
an ambient temperature at the time of mixing in the mixing vessel;
a weight of the batch when the batch is mixed in the mixing vessel;
an ambient relative humidity when the batch is mixed in the mixing vessel;
a slump of the batch at the time of transfer of the batch into a truck vessel on a mixing truck;
a slump of the batch at the time of emptying the batch from the truck vessel into a concrete pumper;
a temperature of the batch at the time the batch is poured into the concrete form;
a duration of a transit interval defined by discharge of the batch from the mixing vessel to the truck vessel and the discharge of the batch from the truck vessel;
a number of rotations of the truck vessel during the transit interval; and
a temperature of the batch in the concrete pumper.

10. The method of claim 9 further comprising:
comparing the weight of the batch in the mixing vessel to an ingredient weight based upon the cement quantity, the water quantity, the sand quantity, and the aggregate quantity; and
where the weight of the batch in the mixing vessel exceeds the ingredient weight, generating an alert to indicate the presence of additional ingredients.

11. The method of claim 1 wherein the performance criterion is selected from a performance criterion group, the performance criterion group consisting of:
air content as defined by ASTM C231;
air content of fresh concrete by volumetric method as defined by ASTM C173;
density, yield, and air content of concrete as defined by ASTM C138;
compressive strength as defined by ACI 318;
compressive strength of concrete by Schmidt hammer as defined by ASTM C805;
penetration resistance as defined by ASTM C803;
ultrasonic pulse velocity as defined by ASTM C597;
pull out test as defined by ASTM C900;
drill core compression as defined by ASTM C42; and
cast in place cylinder compressive strength as defined by ASTM C873.
